(19) United States
(12) Patent Application Publication
(10) Pub. No.: US 2025/0268506 A1
(43) Pub. Date: Aug. 28, 2025

(54) ELECTROCARDIOGRAPHIC SIGNAL MEASURING APPARATUS

(71) Applicant: MAXELL, LTD., Kyoto (JP)

(72) Inventors: Satoshi MATSUNUMA, Kyoto (JP); Reiji HATTORI, Kasuga-shi (JP)

(21) Appl. No.: 18/857,870

(22) PCT Filed: Mar. 23, 2023
(86) PCT No.: PCT/JP2023/011568

§ 371 Date: Oct. 18, 2024

(30) Foreign Application Priority Data
Apr. 19, 2022 (JP) 2022-068912

(51) Int. Cl. A61B 5/318; A61B 5/00; A61B 5/256; A61B 5/277; A61B 5/308

(52) U.S. Cl. CPC A61B 5/318; A61B 5/256; A61B 5/277; A61B 5/308; A61B 5/6805

(57) ABSTRACT

An electrocardiographic signal measuring apparatus includes capacitive coupling type detection electrodes that detect an electrocardiographic signal of a subject without coming into contact with a skin of a body; a first amplifier that amplifies a detection signal detected by the detection electrodes; a feedback circuit that returns an inverted signal obtained by inverting an in-phase signal from the detection electrodes to the body of the subject via a feedback electrode; and guard electrodes placed via an insulator on a side, of the detection electrodes, opposite to a side facing the body of the subject. The detection electrode is connected to one of differential inputs of the second amplifier, the guard electrode is connected to the other of the differential inputs of the second amplifier, and outputs of the second amplifiers are connected respectively to differential inputs of the first amplifier.

### TECHNICAL FIELD

The present invention relates to an electrocardiographic signal measuring apparatus for measuring electrocardiographic signal waveforms of heart beats.

### BACKGROUND ART

In recent years, cardiogenic cerebral infarctions have increased due to global aging and Westernization of dietary habits. The cardiogenic cerebral infarctions is a cerebral infarction in which blood clots formed in the heart are carried to the brain via a blood vessel and occludes the blood vessel in the brain. This cerebral infarction is also called a knock-out cerebral infarction because the occurrence thereof causes severe damage in the brain, and in the worst case, may lead to death. In addition, the ratio of the cardiogenic cerebral infarctions accounts for 20 to 30% among cerebral infarctions, and as the cardiogenic cerebral infarctions occur without a warning as compared with other types of cerebral infarctions, the prevention thereof is difficult. However, it becomes possible to find out a behavior serving as a precursor of the cerebral infarction in the measurement of an electrocardiogram.

One of them is a sign of irregular pulses called auricular fibrillation that is a phenomenon in which a sawtooth wave-like fine continuous waveform is observed on an electrocardiogram. Since auricular fibrillation is transient in most cases, and it is often impossible to confirm atrial fibrillation on an electrocardiogram during an examination, a portable electrocardiograph is mounted for one to several days to examine whether auricular fibrillation occurs or not.

Here, a change in a ST section, which is a section from the end of a S-wave to the beginning of a T-wave on the electrocardiogram, is important for diagnosis. That is, for example, the rise in the ST section is found in epicardial ischemia, myocardial infarction, myocarditis, Brugada syndrome, etc. Conversely, the decrease in the ST section occurs when the blood flow deteriorates in the heart muscle (endocardial ischemia), when the heart muscle becomes thick (cardiac hypertrophy), or the like. When a T-wave that has usually a mountain shape is recessed like a valley, myocardial infarction, cardiac hypertrophy due to high blood pressure or cardiomyopathy, intracerebral bleeding, or the like is suspected.

Such an observation cannot be detected by a general heart rate meter, which is represented by a watch type heart rate meter. A portable electrocardiograph used for such an application is called a Holter electrocardiograph, and is often a small-sized battery-driven recorder having a bipolar type lead-I electrode placement, while a clinical type 12-lead electrocardiograph is for a measurement on a subject in a supine position during a resting period. The electrode has a low polarization voltage, and a stable silver-silver chloride electrode and a conductive gel having excellent adhesion are fixed to a skin with a surgical tape.

In addition, a device with an electric circuit for measuring a potential difference of a bipolar electrode, recording and transmitting an electrocardiographic signal is miniaturized, integrated with a conductive electrode, and directly fixed to a skin with a band (belt) or the like to press other electrodes against a body. One example thereof is a portable electrocardiograph measuring apparatus disclosed in Patent Literature 1.

Furthermore, as an apparatus for measuring an electrocardiogram more easily, a wristwatch-type electrocardiograph disclosed in Patent Literature 2 has been proposed. In this wristwatch type, by bringing a finger of a hand on a side opposite to a wrist wound with the wristwatch into contact with an electrode on the upper surface of the wristwatch, it is possible to perform the electrocardiogram measurement through the standard limb lead-I between the right hand and the left hand.

### CITATION LIST

### Patent Literature

Patent Literature 1: JP 2007-195693 A
Patent Literature 2: JP 60-103935 A

### SUMMARY OF INVENTION

### Technical Problem

However, in the portable electrocardiograph measuring apparatus integrated with a band provided with a plurality of electrode portions as disclosed in Patent Literature 1, it is necessary to press an electrode on the external side of a wound band against the body surface, and thus, a long-term measurement is difficult. In addition, as the pressing pressure and angle fluctuate even for a short period of time, it is difficult to acquire a stable electrocardiogram for a long period of time. In addition, a trace of an electrode or a band is left on a skin portion to be pressed, or a phenomenon in which blood congestion occurs with excessive pressing is also observed. Furthermore, skin inflammation such as a heat rash may occur at a wrist portion wound with a band. In addition, when a subject walks in a standing position or shakes the body, a reference potential of an electrocardiogram becomes unstable, a baseline fluctuates, and thus, it becomes difficult to acquire an accurate electrocardiogram.

In addition, in the wristwatch-type electrocardiograph of Patent Literature 2, it is necessary to bring a finger of a hand opposite to the hand having the wristwatch wound therearound, into contact softly with an electrode portion on the upper surface of the wristwatch, and if the degree of force or the angle of contact deviates, or the hand shakes, the reference potential fluctuates and the baseline of the electrocardiogram fluctuates so that the measurement becomes impossible. In addition, the wristwatch-type electrocardiograph is for measuring an electrocardiogram for a very short time, and it is possible to measure the electrocardiogram only when a subject is in a resting state in a sitting posture. Therefore, if the subject stands up, walks, or moves the body, a contact surface of the finger easily fluctuates so that the measurement becomes impossible. Furthermore, it is impossible to grasp a sudden fluctuation such as irregular pulses in the electrocardiogram for a long period of time.

The present invention is made in view of the above-described circumstances, and aims to provide an electrocardiographic signal measuring apparatus capable of accurately and stably measuring an electrocardiographic signal over a long period of time in a non-contact manner without imposing a burden on a body even in a noisy environment or in a normal living environment such as walking.

### Solution to Problem

To solve the problem described above, the present invention provides an electrocardiographic signal measuring apparatus for measuring an electrocardiographic signal of a subject, the electrocardiographic signal measuring apparatus including:
two capacitive coupling type detection electrodes for detecting the electrocardiographic signal of the subject without coming into contact with a skin of a body;
a first amplifier for amplifying a detection signal detected by the two detection electrodes;
a feedback circuit that returns an inverted signal obtained by inverting an in-phase signal from the two detection electrodes to the body of the subject via a feedback electrode;
an A/D converter that converts the detection signal amplified through the first amplifier into a digital signal;
a communication circuit for transmitting the digital signal outputted from the A/D converter;
two guard electrodes placed via an insulator on a side, of each of the detection electrodes, opposite to a side facing the body of the subject; and
two second amplifiers, wherein
each of the detection electrodes is connected to one of differential inputs of each of the second amplifiers,
each of the guard electrodes is connected to the other of the differential inputs of each of the second amplifiers, and
outputs of the second amplifiers are connected respectively to differential inputs of the first amplifier.

According to the above configuration of the present invention, since two capacitive coupling type detection electrodes for detecting the electrocardiographic signal of the subject without coming into contact with the skin of the body are provided, it is also possible to measure the electrocardiographic signal in a non-contact manner, for example, from above clothes. Therefore, it is possible to measure the electrocardiogram in daily life without imposing a burden on a body. In addition, it is possible to avoid a phenomenon in which a trace of an electrode or the like is formed on a skin portion, inflammation of the skin such as sweat rash is caused, or congestion occurs in which the electrode is pressed too strongly.

According to the above configuration of the present invention, the guard electrode is disposed via the insulator on the detection electrode on the side opposite to the side facing the body of the subject, and the detection electrode and the guard electrode are connected to the respective differential inputs of the second amplifier. Therefore, it is possible to prevent the fluctuation of a floating potential existing around the body, movement of the conductor placed in the periphery, and fluctuation of the baseline of the electrocardiographic signal due to the movement of people present therearound. In addition, it is possible to prevent changes in environmental potential, high-frequency noise, and power supply system noise from surrounding electronic devices and power supply lines from being superimposed on the electrocardiographic signal. That is, according to the above configuration of the present invention, it is possible to measure the electrocardiographic signal stably for a long period of time, and obtain a clear electrocardiographic signal while suppressing noise.

Accordingly, it becomes possible to detect sudden irregular pulses such as auricular fibrillation, which is a sign of a cardiogenic cerebral infarction that requires the long-term observation of an electrocardiogram. In addition, it is also possible to measure the electrocardiogram during walking or rehabilitation in a convalescent stage, while suppressing the influence of noise due to body motion.

In addition, it is preferable that the detection electrode is connected to one of the differential inputs of the second amplifier via a center conductor of a coaxial cable, and the guard electrode is connected to the other of the differential inputs of the second amplifier via an external conductor of the coaxial cable. According to such a configuration, it is possible to more reliably prevent noise associated with changes in environmental potential from being superimposed on a detection signal (electrocardiographic signal), which is advantageous. When the coaxial cable is used, it is more preferable to fix the connecting portion between the center conductor of the coaxial cable and the detection electrode as well as the joint portion between the external conductor and the guard electrode with an insulator such as an insulating epoxy resin. Accordingly, it is possible to prevent a short circuit between the detection electrode and the guard electrode so that the connection between the coaxial cable and the electrode portion can be prevented from being cut off due to the body motion or the like.

In the above configuration, the detection signal from the detection electrode is amplified by the first amplifier, and it is preferable that the potential difference between the two detection electrodes is accordingly amplified to, for example, approximately 100 times. In addition, noise in a frequency domain other than the electrocardiographic signal may be removed by a filter such as a high-pass filter or a low-pass filter on the downstream side of the first amplifier or the like. Here, the filter configured by a high-pass filter, a low-pass filter, or the like may be set so that a signal in a band of, for example, 0.5 to 40 Hz passes.

In addition, according to the above configuration, the feedback circuit that returns an inverted signal obtained by inverting an in-phase signal from the two detection electrodes to the body of the subject via a feedback electrode is provided, and thus, it is possible to easily remove power supply system noise and other unnecessary interference noise inside and outside the measuring apparatus.

In addition, it is preferable that the above-described configuration further includes two network resistors, each of which is configured by connecting a plurality of bias resistors in a T-shape and has three ends including a first end, a second end, and a third end, the first end of each of the network resistors is connected to one of the differential inputs of each of the second amplifiers, and the second end of each of the network resistors is connected to the other of the differential inputs of the second amplifiers.

By increasing the bias resistance by the T-type network resistor, it becomes possible to detect the body potential by the second amplifier having high impedance through capacitive coupling in a state where a subject is wearing the clothing. According to this, it is also possible to shorten the time taken from the over-range of the front end circuit until the voltage returns to a detectable voltage. That is, in the present invention, the input side has a high impedance due to the capacitive coupling between the detection electrode and the body. However, in a case where the second amplifier is configured to cope with this high impedance, the time constant increases, and when an over-range signal enters the front end circuit, the time taken until the voltage returns to the detection voltage is prolonged. During this period of time, a bioelectric potential signal cannot be acquired. In the above configuration of the present invention, by using, for example, a non-linear resistor (varistor) as one of the T-type network resistors and connecting the resistor to the lead-off detection circuit of the first amplifier, when the input-side potential exceeds the varistor voltage, it is possible to recover the input voltage quickly to a detectable voltage, thereby enabling a high-speed recovery of the electrocardiographic signal.

In addition, it is preferable that the above-described configuration further includes a specific circuit capable of switching a state of the third end of the network resistor between a high level and a low level.

Here, the specific circuit refers to, for example, a lead-off detection circuit or a pulse generator, or a circuit including both of them. According to such a configuration, for example, when a sudden signal change such as a lead-off state is applied to the amplifier by utilizing the lead-off detection function of the first amplifier (for example, adjusting the filter cutoff to automatically raise in a downstream filter of the first amplifier at the time of the lead-off detection), a high-speed recovery is enabled by the outputted signal and the pulse generator so that a settling tail does not continue for a long time (so that it does not take a long time until the voltage returns a detectable voltage). For example, in an example of using the first amplifier having a built□in single-lead front end with a lead-off or out-of-range detection function, in the DC lead-off or out-of-range detection function mode, the level of the third end of one of the network resistors becomes a high level when the positive-side electrode is disconnected (by being come off or the like) or is out of range, whereas the level thereof becomes a low level when the electrode is connected. The level of the third end of the other network resistor becomes a high level when the negative-side electrode is disconnected or out of range, whereas the level thereof becomes a low level when the electrode is connected. By instantaneously turning off the low-pass filter in this input by the pulse generator, a high-speed recovery is enabled.

In addition, the above-described configuration may further include a mounting belt that is mounted on the body of the subject in a wound state in a freely attachable and detachable manner and is configured by arranging the two detection electrodes and the feedback electrode.

Furthermore, the above-described configuration may further include a mounting vest that is wearable by the subject on an upper half of the body and is configured by arranging the two detection electrodes and the feedback electrode.

According to this, the subject can mount the mounting belt or the mounting vest alone and the respective electrodes can be positioned at desired positions. Note that, as the mounting belt or the mounting vest, it is preferable to use a medical belt or vest having stretchability. In addition, in a case of using a belt or vest having a brushed surface, it also becomes possible to fix the electrocardiographic measurement device section including the above-described device components other than the electrodes with a hook-and-loop fastener (MAGICTAPE (registered trademark)) or the like, which is convenient. In addition, by providing the mounting belt or the mounting vest with the electrocardiographic measurement device section, the attachability/detachability can be further improved.

### Advantageous Effects of Invention

According to the present invention, it is possible to provide an electrocardiographic signal measuring apparatus capable of accurately and stably measuring an electrocardiographic signal over a long period of time in a non-contact manner without imposing a burden on a body even in a noisy environment or in a normal living environment such as walking.

### BRIEF DESCRIPTION OF DRAWINGS

FIG. 1 is a block diagram illustrating an overall configuration of an electrocardiographic signal measuring apparatus according to one embodiment of the present invention.

FIG. 2 is a detailed structural diagram of a detection electrode used in the electrocardiographic signal measuring apparatus illustrated in FIG. 1.

FIG. 3 illustrates a state in which the electrocardiographic signal measuring apparatus is mounted on a subject with a mounting belt.

FIG. 4 illustrates a state in which the electrocardiographic signal measuring apparatus is mounted on a subject with a mounting vest.

FIG. 5 shows waveforms of electrocardiographic signals in an environment where power supply system noise is large: FIG. 5(a) shows a waveform obtained by the electrocardiographic signal measuring apparatus according to the embodiment in FIG. 1, and FIG. 5(b) shows a waveform of an electrocardiographic signal obtained by a lead-I contact type electrocardiograph according to a comparative example.

FIG. 6 shows waveforms of electrocardiographic signals during walking: FIG. 6(a) shows a waveform obtained by the electrocardiographic signal measuring apparatus according to the embodiment in FIG. 1, and FIG. 6(b) shows a waveform of an electrocardiographic signal obtained by the lead-I contact type electrocardiograph according to the comparative example.

### DESCRIPTION OF EMBODIMENTS

Hereinafter, an embodiment of the present invention will be explained with reference to the drawings, and the present embodiment enables the realization of a highly reliable system, particularly in a sensing system, contributes to the development of a tough infrastructure, and aims for “3. Ensure healthy lives and promote well-being for all at all ages” of Sustainable Development Goals (SDGs) proposed by the United Nations.

Note that, in the present specification, the term “connect” basically includes both a case of being directly connected and a case of being indirectly connected. For example, in a case where “a certain circuit and a certain circuit are connected”, another circuit, an element such as a resistor, or the like may be interposed therebetween. In other words, it is sufficient that a signal is transmittable therebetween.

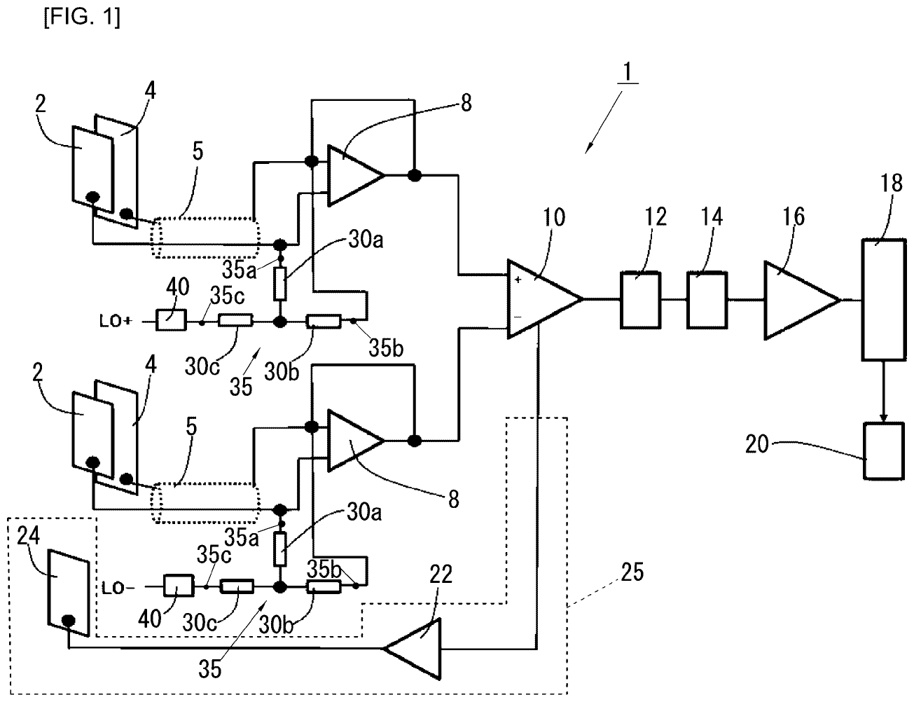

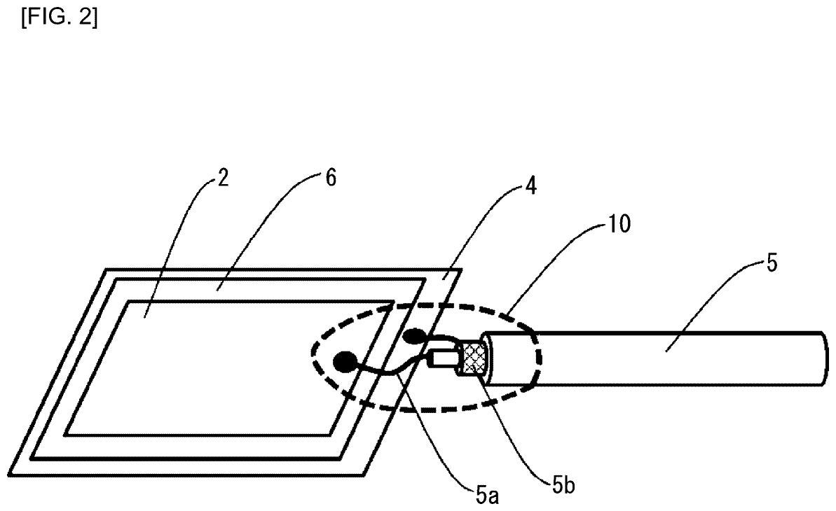

FIG. 1 illustrates an overall configuration of the electrocardiographic signal measuring apparatus 1 according to one embodiment of the present invention for measuring an electrocardiographic signal of a subject. As illustrated in the drawing, the electrocardiographic signal measuring apparatus 1 according to the present embodiment includes two capacitive coupling type detection electrodes 2, 2 for detecting an electrocardiographic signal of a subject without coming into contact with a skin of the body (it is possible to measure an electrocardiographic signal from above clothes), and two guard electrodes 4, 4 for active guarding placed on a side, of each detection electrode 2, opposite to a side facing the body of the subject via an insulator 6 (insulating film, see FIG. 2). Each detection electrode 2, 2 is electrically connected to one of differential inputs of a second amplifier (front end amplifier) 8 corresponding thereto via a center conductor 5a (see FIG. 2) of a coaxial cable 5, and each guard electrode 4, 4 is electrically connected to the other of the differential inputs of the second amplifier 8 corresponding thereto via an external conductor 5b (see FIG. 2) of the coaxial cable 5. Note that in these two second amplifiers 8 provided corresponding to the two detection electrodes 2, 2 (guard electrodes 4, 4), an output and an input to which the guard electrode 4 is connected are connected. In addition, a connecting portion between the center conductor 5a of the coaxial cable 5 and the detection electrode 2, and a connecting portion between the external conductor 5b of the coaxial cable 5 and the guard electrode 4 are respectively fixed with an insulating epoxy resin 10 so as to maintain the insulation property. As described above, when a laminate structure having a thin insulating film 6 sandwiched between the detection electrodes 2, 2 and the guard electrodes 4, 4 is configured, an effect of being hardly affected by the potential and a power supply line around the body, is obtained. In addition, by fixing the connecting portion between the center conductor 5a of the coaxial cable 5 and the detection electrode 2, and the connecting portion between the external conductor 5b of the coaxial cable 5 and the guard electrode 4 with the insulating epoxy resin 10, it is possible to avoid a short circuit between the detection electrode 2 and the guard electrode 4 and prevent the coaxial cable 5 and the electrodes 2 and 4 from being disconnected due to the body motion or the like.

In addition, the electrocardiographic signal measuring apparatus 1 of the present embodiment also includes a first amplifier (instrumentation amplifier) 10 for amplifying a detection signal detected by the two detection electrodes 2, 2 (amplifying a potential difference between the two detection electrodes 2, 2). Outputs of the two second amplifiers 8, 8 described above are connected to the two differential inputs of the first amplifier 10, respectively. Note that, in the present embodiment, a potential difference between the two detection electrodes 2, 2 in the first amplifier 10 is amplified up to approximately 100 times. In addition, the potential difference between the detection electrodes 2, 2 is one to several mV in the case of a healthy person.

In addition, the output of the first amplifier 10 is connected to, for example, a high-pass filter 12 having a cutoff frequency of 0.5 Hz for removing noise (noise components other than the electrocardiographic signal) from an output signal outputted from the first amplifier 10, and this high-pass filter 12 is connected to, for example, a low-pass filter 14 having a cutoff frequency of 40 Hz. That is, the electrocardiographic signal measuring apparatus 1 includes a filter circuit for removing noise in a predetermined band from the detection signal.

In addition, the low-pass filter 14 is connected to a digital communication unit 20 serving as a communication circuit for transmitting a digital signal, via a third amplifier (operational amplifier) 16 for further amplifying a signal from the filter, for example, 11 times and an A/D converter 18 for converting an analog signal into a digital signal. Note that the A/D converter 18 digitizes the analog output of 0 to 3.36 V with 14 bits at a time resolution of, for example, 250 samples/second. In addition, the digital communication unit 20 can transmit a digital signal to a predetermined electronic equipment in accordance with a predetermined telecommunications standard such as Bluetooth (registered trademark). For example, the measured electrocardiographic signal is transmitted by the digital communication unit 20 to a smartphone, a personal computer (PC), or the like through wireless communications such as Bluetooth or WIFI, and an autonomic nervous system index can be calculated through heart rate analysis, time analysis, frequency analysis, or nonlinear analysis of fluctuation or the like (by using an application in the smartphone or the PC) on the smartphone or the PC. In addition, it is possible to upload an electrocardiographic signal, a heart rate obtained from the electrocardiographic signal, results of time analysis or frequency analysis based on the electrocardiographic signal, and the like to a cloud computer via the Internet, and store and analyze the data.

The electrocardiographic signal measuring apparatus 1 of the present embodiment includes a feedback circuit 25 that returns an inverted signal obtained by inverting an in-phase signal from the two detection electrodes 2, 2 to the body of the subject via a feedback electrode 24. Specifically, the feedback circuit 25 includes a fourth amplifier 22 for returning an inverted signal obtained by inverting the in-phase signal from the detection electrodes 2, 2 outputted from the first amplifier 10 to the feedback electrode 24, and the feedback electrode 24 electrically connected to the output of the fourth amplifier 22. The fourth amplifier 22 can be regarded as an amplifier that drives the feedback electrode 24. Note that the inversion of the in-phase signal from the detection electrodes 2, 2 may be performed by the first amplifier 10 or may be performed by the fourth amplifier 22.

In addition, the electrocardiographic signal measuring apparatus 1 of the present embodiment has two network resistors 35, 35, respectively corresponding to two detection electrodes 2, 2 (guard electrodes 4, 4), each of which is configured by connecting a plurality of (three in the present embodiment) resistors 30 (30a, 30b, 30c) in T-shape and has three ends of a first end 35a, a second end 35b, and a third end 35c. In this case, the first end 35a of each network resistor 35 is connected to one of the differential inputs of the corresponding second amplifier 8, and the second end 35b of each network resistor 35 is connected to the other of the differential inputs of the corresponding second amplifier 8. Note that, in consideration of an input impedance of the second amplifier 8, a frequency band for detecting electrocardiographic waveforms, or the like, each resistor 30 preferably has, for example, several hundred MΩ to several TΩ. In the present embodiment, a resistor of 10 MΩ was used for the resistor 30a, a resistor of 1 kΩ was used for the resistor 30b, and a non-linear resistor 30c which has 250 MΩ up to a varistor voltage of 8 V was used for the resistor 30c. Under this condition, the input impedance becomes 2.5 TΩ until the input-side voltage reaches the varistor voltage, and thus, a potential difference on the body surface can be measured by the capacitive coupling type electrode, with the second amplifier which supports high impedance. As described above, a part or all of the resistors constituting the network resistor 35 may be non-linear resistors (varistors), or fixed resistors, or other types of resistors. In addition, each of the resistors 30a, 30b, and 30c may be realized by a plurality of resistance elements or the like.

The electrocardiographic signal measuring apparatus 1 of the present embodiment also includes a specific circuit capable of switching the state of the third end 35c of each network resistor 35 between a high level and a low level. In particular, in the present embodiment, this specific circuit includes a lead-off detection circuit and a pulse generator 40. For this purpose, in the present embodiment, an instrumentation amplifier with a DC lead-off or out-of-range detection function is used as the first amplifier 10, and the LO+ output at the time of the disconnection or out-of-range of a positive-side electrode, and the LO− output at the time of the disconnection or out-of-range of the negative-side electrode, are connected via the pulse generator 40 as illustrated in FIG. 1, whereby the low-pass filter associated with the first amplifier 10 is instantaneously turned off to minimize the phenomenon that a tail is prolonged when the detection potential suddenly has changed. Here, the lead-off detection circuit can be regarded as a circuit that outputs a high level (or a low level) from a LO+ terminal at the time of the disconnection or out-of-range of the positive-side electrode, and outputs a low level (or a high level) from the LO+ terminal during the normal operation (when the electrode is connected and a normal signal is inputted), or a circuit that outputs a high level (or a low level) from a LO− terminal at the time of the disconnection or out-of-range of the negative-side electrode, and outputs a low level (or a high level) from the LO− terminal during the normal operation (when the electrode is connected and a normal signal is inputted).

Note that, in the present embodiment, the high level is 3 V and the low level is 0 V (ground).

Note that the third end 35c of each network resistor 35 may have a fixed potential. Specifically, the third end 35c may be fixed to, for example, a low level (for example, may be connected to the ground), or a predetermined voltage may be applied thereto. In other words, the electrocardiographic signal measuring apparatus 1 may not include one or both of the lead-off detection circuit and the pulse generator 40.

Note that the configuration of respective circuits and the placement relation of respective circuits in the electrocardiographic signal measuring apparatus 1 may be different from those illustrated in FIG. 1 as long as the various functions described above can be realized. In addition, a part of the various functions (respective circuits) described above may not be present.

In the present embodiment, examples of raw material of the detection electrode 2 include a braided body of conductive fibers produced by plating a polymer synthetic fiber with a metal such as silver, gold, copper, or nickel, and a conductive foam produced by forming the conductive fibers in a nonwoven fabric form. In the case of the conductive foam, the thickness thereof is preferably 0.1 to 5 mm, but in consideration of fitting to the body and clothing, approximately 1 mm is suitable.

In addition, in order to maintain the conduction at a joint portion with the center conductor 5a or the external conductor 5b of the coaxial cable 5 in a favorable state, it may be preferable to have the conductivity not only in the plane direction, but also in the thickness direction. The surface resistance value is desirably 0.01 to 0.1Ω/□, more desirably approximately 0.05Ω/□. In addition, the volume resistance is desirably 15 to 0.1 mΩ, but 1 to 5 mΩ is sufficient. The detection electrode 2 may have a multilayer structure in view of the strength and weight in addition to the resistivity. In the present embodiment, a conductive foam (surface resistance value: 0.055Ω/□, volume resistance value: 4 mΩ) having a thickness of 1 mm and using copper-plated fibers is cut into a size of 3 cm×5 cm to obtain the detection electrode 2.

Alternatively, as the detection electrode 2, a conductive metal such as a stainless alloy, a copper plate, silver, gold, or platinum may be used. The thickness in this case is preferably such a thickness that bending thereof through contact with the body can be avoided, and is approximately 0.1 to 1 mm, for example. The periphery of the detection electrode 2 needs to be appropriately chamfered. Furthermore, as the detection electrode 2, conductive rubber, conductive plastic, or the like may be used. The surface resistance becomes higher than that of a conductive fiber or a metal plate in this case, and thus, it is desirable to lower the surface resistance by, for example, applying a silver paste to the surface thereof.

Although a polyimide film having a thickness of 0.066 mm is used as the insulating film 6 in the present embodiment, polyester, mica, fluororesin, polypropylene, or the like may be used in addition to the polyimide film. The thickness thereof is preferably 0.01 to 1 mm. The insulating film 6 needs to be joined with a strong adhesive tape or adhesive so as not to be charged by friction with the detection electrode 2 and the guard electrode 4. Although a conductive foam, the material and the size of which are identical to the detection electrode 2, is used as the guard electrode 4 in the present embodiment, a film for electromagnetic shielding such as a copper foil film may also be used. Furthermore, a meshed metal net can be used. Although the detection electrode 2 and the center conductor 5a of the coaxial cable 5, and the guard electrode 4 and the external conductor 5b of the coaxial cable 5 are respectively joined by using a conductive adhesive in the present embodiment, a conductive hook, soldering, or the like may also be used.

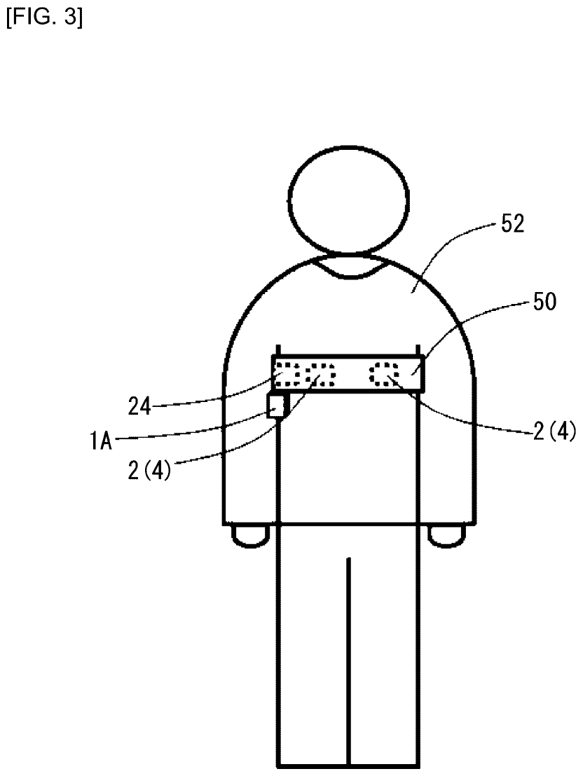

In the configuration described above, during the measurement, the detection electrode 2 and the guard electrode 4 to be integrated in a state of being insulated from each other, are mounted and fixed (wound and mounted), in a freely attachable and detachable manner, to a subject 52 wearing clothes (for example, a T-shirt) using a mounting belt 50 having a width of 4.5 cm and a length of 120 cm, for example, as illustrated in FIG. 3. In this case, the mounting belt 50 has, for example, an inner side (subject 52 side: clothing side) made of natural rubber and an outer side made of nylon (which is, for example, brushed) and is sufficiently flexible and stretchable. In addition, the detection electrode 2 and the guard electrode 4 are preferably attached in a freely attachable and detachable manner, and are more preferably attached such that the position thereof is adjustable, to the inner side (inner surface) of the mounting belt 50 facing the clothing side of the subject 52 in the mounted state. In addition, the feedback electrode 24 is attached to the inside of the mounting belt 50 preferably in a freely attachable and detachable manner and/or in a position adjustable manner, and an electrocardiographic measurement device section 1A including all components of the electrocardiographic signal measuring apparatus 1 except for the electrodes 2, 4, and 24 is attached to a nylon portion (for example, the lateral surface) on the outside in a freely attachable and detachable manner and/or in a position adjustable manner by, for example, a hook-and-loop fastener. Then, in the mounting state, illustrated in FIG. 3, with the mounting belt 50 configured by arranging the electrodes 2, 4, and 24 (state where the mounting belt 50 is wound around the chest of the subject 52 from above clothes), the two detection electrodes 2, 2 (guard electrodes 4, 4) are respectively placed under the nipples of the left breast and the right breast of the subject 52, and the feedback electrode 24 is placed under the right arm of the subject 52. Obviously, the positions of the electrodes 2, 4, and 24 are not limited thereto, and may be positioned at a desired site.

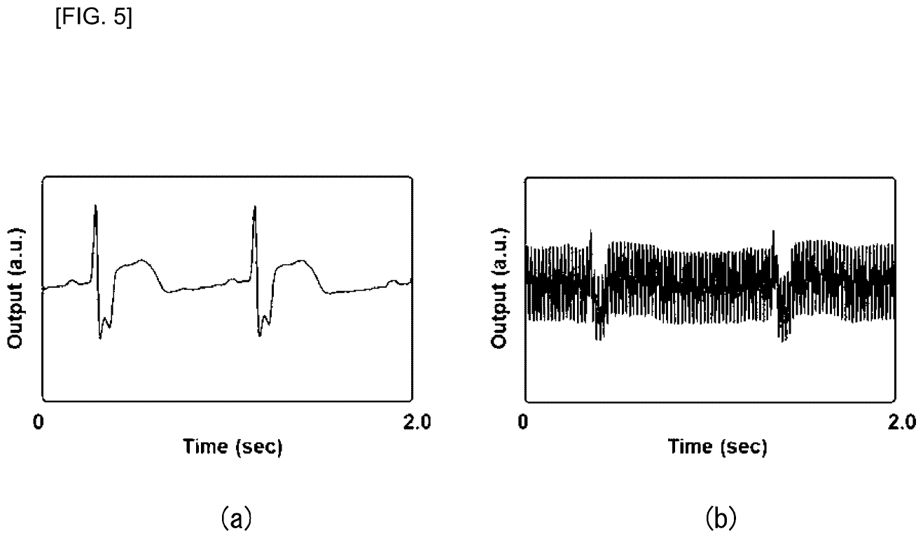

In addition, with such electrode placement, the electrocardiographic signal of the subject 52 was measured using the electrocardiographic signal measuring apparatus 1 in an environment where there seems to be much power supply system noise. That is, the subject 52 was asked to sit near three personal computers powered from an alternating-current power outlet, and the electrocardiographic signal was measured to obtain an electrocardiogram as shown in FIG. 5(a). In the drawing, the horizontal axis represents a waveform for two seconds. As can be seen from this drawing, favorable waveforms without power supply system noise, that is, two P□waves, QRS□waves, and T□waves in the electrocardiogram are clearly observed.

As a comparative example, FIG. 5(b) shows a waveform measured through the lead-I of a conventional contact type electrocardiograph in the same environment. In this comparative example, Ag/AgCl sticking-type electrodes were mounted on the left and right wrists of the subject. The feedback electrode was brought into contact with the left arm. As is obvious from FIG. 5(b), in the conventional contact type electrocardiograph, the power supply system noise at 50 Hz becomes large in this environment, and it is impossible to accurately observe the electrocardiographic waveform.

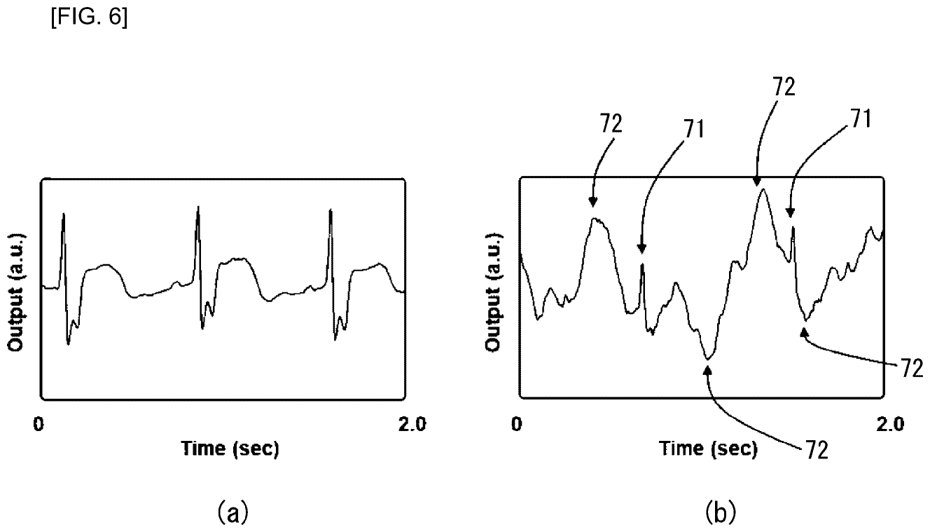

Furthermore, the electrocardiographic signal measuring apparatus 1 of the present embodiment was mounted on the subject 52 as illustrated in FIG. 3, and the electrocardiographic waveform was measured while making the subject 52 walk on an outdoor pavement. The obtained signal was transmitted to a smartphone through Bluetooth communications, and a waveform as illustrated in FIG. 6(a) was obtained. As can be seen from this waveform, it is possible to clearly observe a P-wave, a QRS-wave, and a T-wave even during walking, and the fluctuation of a baseline due to the walking hardly affects thereon.

As a comparative example, FIG. 6(b) illustrates an example in which the measurement is performed during walking with a conventional contact type electrocardiograph in the same manner. As is obvious from this drawing, although a portion 71 corresponding to the R wave of the electrocardiographic signal is observed in the contact type, a fluctuation 72 resulting from the body motion based on running larger than that is observed, and thus it is difficult to perform an analysis accurately.

As can be seen from the above results, according to the electrocardiographic signal measuring apparatus 1 of the present embodiment, the electrocardiographic waveform can be measured from above clothes by a capacitive coupling mechanism by a high impedance front end, and moreover, it becomes possible to measure the electrocardiographic signal accurately without superimposition of noise even in an environment where power supply system noise is too large to perform measurement by an electrocardiograph using a conventional sticking type electrode. In addition, according to the electrocardiographic signal measuring apparatus 1 of the present embodiment, even when the body motion is large, for example, during walking and a fluctuation becomes too large to perform the measurement thereon by the electrocardiograph using a conventional sticking type electrode, a baseline becomes stable and the electrocardiographic waveform can be clearly observed.

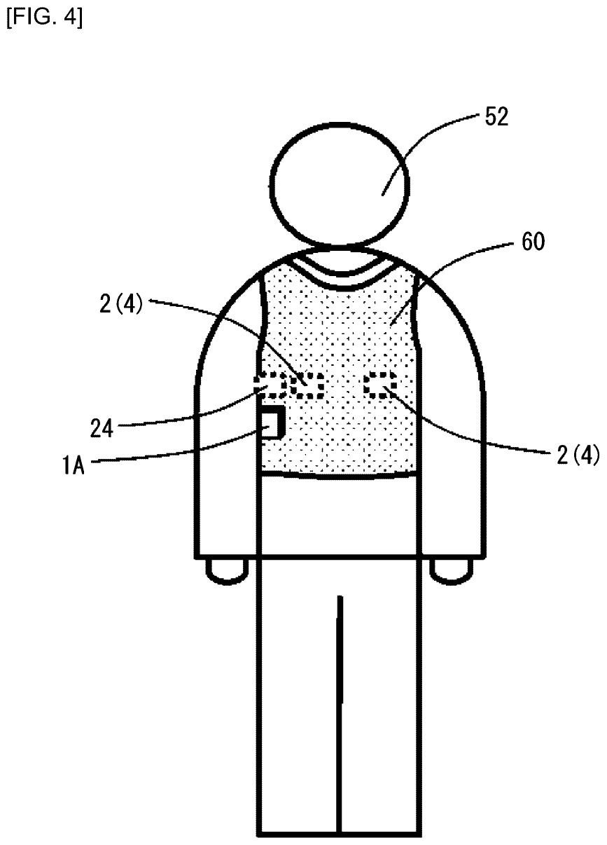

FIG. 4 illustrates a modification of the form of mounting the electrocardiographic signal measuring apparatus 1 to the subject 52. In this modification, each of the electrodes 2, 4, and 24 is positioned with respect to the body of the subject 52 not by the mounting belt, but by the mounting vest 60 that is wearable by the subject 52 on an upper half of the body. In this case, the mounting vest 60 configured by arranging the electrodes 2, 4, and 24, is made of a stretchable cloth such as a knit cloth, a fleece cloth of polyethylene terephthalate, or a pile cloth providing no feeling of pressure even when being in close contact with the body, and can be put on from the neck and through the sleeves from above clothes such as a T-shirt or an underwear, whereby it is easily wearable. In addition, since the two detection electrodes 2, 2 and the feedback electrode 24 are fixed respectively to optimal locations, it is suitable for a situation where easy and quick mounting of the electrocardiographic signal measuring apparatus 1 is desirable.

Also in this case, the electrodes 2, 4, and 24 are preferably attached to the mounting vest 64 in a freely attachable and detachable manner and/or in a position adjustable manner, and the electrocardiographic measurement device section 1A including all components of the electrocardiographic signal measuring apparatus 1 except for the electrodes 2, 4, and 24 is attached to the outside of the mounting vest 60 in a freely attachable and detachable manner and/or in a position adjustable manner by, for example, a hook-and-loop fastener. A lidded attachment portion, such as a pocket, may be provided in advance on the mounting vest 60 to enable the attachment in a freely attachable and detachable manner and/or in a position adjustable manner. In addition, in the mounting state, illustrated in FIG. 4, with the mounting vest 60, the two detection electrodes 2, 2 (guard electrodes 4, 4) are respectively placed under the nipples of the left breast and the right breast of the subject 52, and the feedback electrode 24 is placed under the right arm of the subject 52. Obviously, the positions of the electrodes 2, 4, and 24 are not limited thereto, and may be positioned at a desired site. Since such a vest type mounting body provides a high fit feeling with respect to the body and is hardly displaced, it is suitable for light exercises such as walking, rehabilitation in a convalescent stage, a long-term measurement in a horizontal position on a bed, and the like.

In addition, the electrocardiographic signal measuring apparatus 1 was mounted on the subject 52 using such a mounting vest 60 from above a T-shirt, and the electrocardiographic signal was measured while making the subject 52 walk on the outdoor pavement. Although not illustrated, the obtained electrocardiographic waveform was equivalent to an electrocardiographic signal waveform in FIG. 6(a) described above. There was no superimposition of environmental noise, a fluctuation of a baseline due to the body motion accompanied with walking, and the like. In addition, even when the subject was asked to continue fast walking on a treadmill for 30 minutes with the speed set to 6 km/h, the electrocardiographic signal could be measured clearly.

Furthermore, an attempt was made to detect irregular pulses while the subject wearing the mounting vest 60 was sleeping at night. The subject was a 72 year old male with a medical history of diabetes and high blood pressure. The subject wore the mounting vest 60 above a thick underwear and fell asleep in a supine position on a bed for 7 hours. As a result, an irregular pulse, which is considered to be a paroxysmal atrial fibrillation, was observed once. It has been found that the risk of onset of cardiogenic embolism due to a paroxysmal atrial fibrillation is equivalent to that of a persistent auricular fibrillation, and it has been revealed that a paroxysmal atrial fibrillation without subjective symptoms can be found out by a long-term measurement using the electrocardiographic signal measuring apparatus 1 according to the present embodiment.

As described above, according to this modification, the right and left detection electrodes 2, 2 (guard electrodes 4, 4) and the feedback electrode 55 can be easily arranged at desired positions on the chest simply by wearing the mounting vest 60, and thus, it is possible to suppress a burden on the subject 52 when the electrocardiographic signal is observed even during sleeping or over a long period of time. In addition, it is possible to measure the electrocardiographic signal even in daily life, during light work, and exercise without behavior restrictions. Accordingly, it is possible to significantly increase the quality of life (QOL) of the subject 52.

Note that a meshed cloth or a pile cloth can be used for the mounting vest 60 for a hot season such as summer. Furthermore, a cloth providing contact cold sensation may be used, such as a core-sheath composite fiber using polyester for a core and EVOH (composite resin of a polyethylene resin and a polyvinyl alcohol resin) for a sheath. In addition to the vest shape, an X shape, a training bib-like shape, or a number cloth-like shape may be used. The detection electrode 2 and the feedback electrode 24 may be positioned not only on the chest, but also at vertical positions on the chest and in the pit of the stomach. Furthermore, although the intensity of the electrocardiographic signal becomes weak, the detection thereof is possible by installing an electrode on the back or the like.

Note that the present invention is not limited to the above-described embodiments, and various modifications can be made without departing from the gist thereof. For example, in the present invention, a form of mounting the electrodes and the electrocardiographic measurement device section to a subject is not limited to the belt or the vest. In addition, a form of configuring the network resistor and the feedback circuit is not limited to the form in the above-described embodiments. In addition, the whole or a part of the above-described embodiments may be combined without departing from the gist of the present invention, or a part of the configuration may be omitted from one of the above-described embodiments.

### REFERENCE SIGNS LIST

1 Electrocardiographic signal measuring apparatus
2 Detection electrode
4 Guard electrode
5 Coaxial cable
5a Center conductor
5b External conductor
8 Second amplifier
10 First amplifier
18 A/D converter
20 Digital communication unit (communication circuit)
24 Feedback electrode
25 Feedback circuit
30a Resistor
30b Resistor
30c Resistor
35 Network resistor
35a First end
35b Second end
35c Third end
50 Mounting belt
60 Mounting vest

1. An electrocardiographic signal measuring apparatus for measuring an electrocardiographic signal of a subject, the electrocardiographic signal measuring apparatus comprising:
two capacitive coupling type detection electrodes for detecting the electrocardiographic signal of the subject without coming into contact with a skin of a body;
a first amplifier for amplifying a detection signal detected by the two detection electrodes;
a feedback circuit that returns an inverted signal obtained by inverting an in-phase signal from the two detection electrodes to the body of the subject via a feedback electrode;
an A/D converter that converts the detection signal amplified through the first amplifier into a digital signal;
a communication circuit for transmitting the digital signal outputted from the A/D converter;
two guard electrodes placed via an insulator on a side, of each of the detection electrodes, opposite to a side facing the body of the subject; and
two second amplifiers, wherein
each of the detection electrodes is connected to one of differential inputs of each of the second amplifiers,
each of the guard electrodes is connected to the other of the differential inputs of each of the second amplifiers, and
outputs of the second amplifiers are connected respectively to differential inputs of the first amplifier.

2. The electrocardiographic signal measuring apparatus according to claim 1, further comprising
two network resistors, each of which is configured by connecting a plurality of bias resistors in a T-shape and has three ends including a first end, a second end, and a third end, wherein
the first end of each of the network resistors is connected to one of the differential inputs of each of the second amplifiers, and
the second end of each of the network resistors is connected to the other of the differential inputs of each of the second amplifiers.

3. The electrocardiographic signal measuring apparatus according to claim 2, further comprising
a specific circuit capable of switching a state of the third end of the network resistor between a high level and a low level.

4. The electrocardiographic signal measuring apparatus according to claim 1, wherein
the detection electrode is connected to one of the differential inputs of the second amplifier via a center conductor of a coaxial cable, and
the guard electrode is connected to the other of the differential inputs of the second amplifier via an external conductor of the coaxial cable.

5. The electrocardiographic signal measuring apparatus according to claim 1, further comprising
a mounting belt that is mounted on the body of the subject in a wound state in a freely attachable and detachable manner and is configured by arranging the two detection electrodes and the feedback electrode.

6. The electrocardiographic signal measuring apparatus according to claim 1, further comprising
a mounting vest that is wearable by the subject on an upper half of the body and is configured by arranging the two detection electrodes and the feedback electrode.
